(19) United States
(12) Patent Application Publication
(10) Pub. No.: US 2013/0225800 A1
(43) Pub. Date: Aug. 29, 2013

(54) NUCLEIC ACID EXTRACTION METHOD

(71) Applicant: David Squirrell, Wiltshire (GB)

(72) Inventor: David Squirrell, Wiltshire (GB)

(73) Assignee: ENIGMA DIAGNOSTICS LIMITED, Wiltshire (GB)

(21) Appl. No.: 13/820,186

(22) PCT Filed: Sep. 1, 2011
(86) PCT No.: PCT/GB2011/051637

§ 371 Date: May 15, 2013

(30) Foreign Application Priority Data
Sep. 3, 2010 (GB) 1014662.9

(51) Int. Cl. C12N 15/10

(52) U.S. Cl. CPC C12N 15/1013
USPC 536/25.42; 422/527

(57) ABSTRACT

There is provided a method of extracting a nucleic acid analyte from a cell or virus in a sample chamber, comprising a) adding disruption beads comprising external silica or glass to the sample chamber; b) agitating the disruption beads within the sample chamber to disrupt the cell; c) adding binding particles comprising external silica or glass to the sample chamber in the presence of a chaotropic agent; d) contacting the contents of the sample chamber with a removal device with which the binding particles reversibly associate; and e) separating the removal device and associated binding particles from the sample chamber, thereby removing the nucleic acid analyte from the sample. There are also provided apparatus and kits for use with the method.

### FIELD OF THE INVENTION

The invention relates to a method for extracting a nucleic acid analyte from cells in a sample. The invention is particularly suitable for samples resistant to existing methods, for example, a sample containing spores of a fungus such as Aspergillus fumigatus.

### BACKGROUND

Laboratory-based methods for lysing spores include the use of equipment such as sonicators, bead beaters, or “French” presses. WO2009/129236 describes extracting nucleic acids from bacterial samples by use of disrupting beads and metal or metal oxide magnetic binding particles. This type of equipment, however, is unsuited for non-laboratory applications where “point-of-care” or “pen-side” automated PCR instruments may be used. Such instruments commonly use a process known as the Boom method (Boom et al. (1990) J. Clin. Microbiol. vol 28 pp 495-503) where cells are lysed using a chaotropic reagent such as guanidinium hydrochloride (GuHCl). The chaotropic agent acts both to lyse the sample and to effect the binding of nucleic acids to silica capture surfaces. In tests on Bacillus bacterial spores this releases only about 1% of the nucleic acid and for Aspergillus fungal spores no measurable nucleic acid is released. Even sonication with GuHCl has been found to be ineffective.

Spores of bacterial and fungal species such as these are environmentally resistant dispersal forms and are of interest as the targets of detection methods. They may be pathogenic to plants, animals or humans, be the cause of unwanted degradation of materials or foodstuffs or cause food poisoning through the generation of toxins (such as aflatoxin produced by Aspergillus flavus). Methods developed to release nucleic acid from resistant spores may also have application to other resilient samples such as ear-punches from farm animals or tissue samples that need to be broken up to release the nucleic acids from an infecting pathogen or tumour cells.

Aspergillus fumigatus is a thermophilic mould which is of concern as a lung pathogen. The spores from this organism are particularly resilient to existing nucleic acid extraction methods. Improvements in the ability to detect A. fumigatus in aerosol samples by nucleic acid-based techniques such as by PCR are of value and importance. A method combining bead beating (vigorous shaking with glass beads) combined with use of AL Buffer (which contains GuHCl, available from Qiagen) and Proteinase K was able to detect as few as 10 Aspergillus conidia per ml in a liquid sample (Griffiths et al. (2006) J. Med. Microbiol. vol 55 pp 1187-1191).

Sample preparation in some instruments (for example, the FL machines from Enigma Diagnostics Ltd (see WO2005/019836), or the machine marketed as “Maxwell®” by Promega Corporation) involves use of a sheathed magnet to transfer magnetic silica microparticles through various processing steps. The inventors have found that such apparatus can also be utilised in a new and effective method that has been shown to release nucleic acids suitable for PCR from even the most resilient spore forms.

### SUMMARY OF INVENTION

According to a first aspect of the invention, there is provided a method of extracting a nucleic acid analyte from a cell or virus in a sample chamber, comprising
a) adding free disruption beads to the sample chamber, the beads optionally comprising external silica or glass;
b) agitating the disruption beads within the sample chamber to disrupt the cell;
c) adding free binding particles comprising external silica or glass to the sample chamber in the presence of a chaotropic agent;
d) contacting the contents of the sample chamber with a removal device with which the binding particles reversibly associate; and
e) separating the removal device and associated binding particles from the sample chamber, thereby removing nucleic acid analyte from the sample.

Reference in this specification to “free” beads and particles indicates that they are not immobilised on a surface and are able to move around within the sample chamber, for example, during agitation step (b). The chaotropic agent may be, for example, guanidinium thiocyanate, guanidinium isothiocyanate or guanidinium hydrochloride, for example present at a final concentration of around 2-4M in the sample chamber. The term “comprising external silica or glass” means a particle or bead made from or at least partially coated with any material comprising silica and/or glass. Therefore, at least some of the external surface of the particle or bead comprises silica and/or glass.

The binding particles comprising external silica or glass are capable of forming a complex with the nucleic analyte when in the presence of the chaotropic agent.

Optionally, steps (a) and (c) can be combined so that the disruption beads, the binding particles and the chaotropic agent are all present when step (b) is carried out. These reagents may be added before or after the sample is placed in the chamber; for example, the chamber may be manufactured so that these reagents are present in the chamber prior to use. The chaotropic agent such as a guanidinium salt has a two-fold effect of lysing cells or virus particles and promoting the binding of the released nucleic acid to the surface of the binding particles.

The disruption beads may be formed by a ceramic material, zirconium, titanium or steel, or any other suitable hard and durable substance, particularly silica or glass. The term “glass”, as used throughout this specification, indicates an amorphous or non-crystalline material comprising silica (SiO₂), typically at about 75% w/w, in combination with one or more other additives, for example, sodium oxide (Na₂O) and/or calcium oxide (CaO). As is well known to the skilled person, glass is an amorphous material that exhibits a transition from a hard, brittle, solid state to a molten liquid state when heated to a sufficiently high temperature, i.e., it exhibits a glass transition. Reference in this specification to a silica material is intended to refer to a material wholly or substantially formed by silica, i.e., silica may be present in the material at a level of at least about 50% w/w, for example about 55%, 60%, 65%, 70%, 75%, 80%, 85%, 90%, 91%, 92%, 93%, 94%, 95%, 96%, 97%, 98% or about 99% w/w. In some embodiments, the “silica material” may be formed by 100% w/w silica.

The agitation in step (b) may comprise grinding the free disruption beads in the chamber using a grinding element or device, in a manner analogous to use of a pestle and mortar, i.e., the grinding element acts to crush and grind the free beads and the cell(s) in the sample against one another and against the sides of the sample chamber, to assist with disruption of the structure of the cell. Alternatively or additionally, the agitation may be facilitated by use of a sonicating element, the sonication primarily acting to cause vigorous movement of the beads rather than necessarily to directly cause disruption to cells in the sample. The grinding element and sonicating element may each be, for example, an elongate member such as a rod, stick or “wand” which can be introduced into the sample chamber, in a manner analogous to a pestle being introduced into a mortar. The grinding element and sonicating element may be combined and may each also act as the removal device in step (d) of the method. Therefore, in some embodiments, steps (a) and (c) can be combined and/or steps (b) and (d) can be combined.

The nucleic acid analyte may be any kind of nucleic acid, for example DNA or RNA and may be naturally occurring in the cell or have been artificially introduced into the cell prior to the operation of the method of the invention. The cell may be a bacterial or a fungal cell, for example, an Aspergillus fumigatus cell. The cell may also be a part of an animal or plant tissue and may also be taken to mean a virion, or virus particle. The cell may be contained within a liquid sample such as an environment sample, or blood, serum, sputum or any other bodily fluid, or within a solid or semi-solid sample such as a soil sample, a foodstuff or a tissue sample obtained from an animal, for example, a tissue biopsy from a human being.

The term “binding particle comprising external silica or glass” means a particle or bead made from or at least partially coated with any solid phase binding material comprising silica and/or glass, which, in the presence of chaotropic reagents, can interact with and adsorb nucleic acids. Therefore, at least some of the external surface of the particle comprises silica and/or glass. The nucleic acid is sufficiently strongly bound that, as the binding particle is removed from the sample chamber in step (e) of the method, the analyte remains attached to the binding particles. The binding of the analyte to the binding material is reversible such that the analyte can be freed from the binding material after step (e), for further chemical or physical processing. Any suitable means can be used for removing the analyte from the binding material including warming, or changing the pH or ionic milieu.

In a preferred embodiment, the disruption beads are glass beads. In a further preferred embodiment, the binding particles are magnetic silica beads and the removal device is a magnet. The inventors were surprised to find that the combined use of glass beads as disruption beads and silica beads as binding particles did not result in significant reduction of the yield of nucleic acid from the sample. This was surprising because the anticipated interaction of nucleic acids with the glass beads in the presence of a chaotropic agent was expected to reduce, by competition, the interaction between the nucleic acids and the silica beads.

The relative packed volumes of the 0.5 mm diameter glass beads and 1 micron magnetic beads used, 500 μl versus 10 μl, respectively, would intuitively suggest a much larger surface area for the glass beads which could compete disadvantageously for the binding of the extracted nucleic acid. However, the surface area to volume ratio of a 1 micron sphere is so much greater than a 500 micron sphere that, treated as spheres, the glass beads and magnetic particles would have almost exactly the same total surface areas of 6,000 square millimetres. Without wishing to be bound by theory, it appears that the surface of the glass beads is smooth and provides a smaller overall surface area than the magnetic particles which, as the result of roughness of the surface resulting from their manufacture using a colloidal process, thereby have a much greater effective surface area. This may provide an explanation for the negligible degree of interference of binding by nucleic acids to the magnetic particles, in the presence of the glass disruption beads. The glass beads may have diameter 0.1-1 mm, preferably about 0.3 mm, 0.4 mm, 0.5 mm, 0.6 mm, or about 0.7 mm. The magnetic silica beads may have diameter 0.1-5 μm, preferably about 0.8 μm, 0.9 μm, 1.0 μm, 1.1 μm or about 1.2 μm.

According to a second aspect of the invention, there is provided apparatus for use in a method of extracting a nucleic acid analyte from a cell in a sample, preferably according to the first aspect of the invention, comprising (a) a first chamber containing free disruption beads (which may comprise external silica or glass) and (b) a chaotropic agent and free binding particles comprising external silica or glass, wherein the chaotropic agent and/or free binding particles are contained in the first chamber and/or in a second chamber included in the apparatus. The apparatus may be a sample cartridge suitable for use, for example, with the FL machines from Enigma Diagnostics Ltd, or the machine marketed as “Maxwell®” by Promega Corporation. For example, the apparatus may be a cartridge or sample platform as shown in FIG. 3 of WO2005/019836.

The chaotropic agent is preferably guanidinium thiocyanate, guanidinium isothiocyanate or guanidinium hydrochloride. The disruption beads may be glass beads or silica beads. The binding particles may be paramagnetic silica beads. When the disruption beads are glass beads, they may be of diameter 0.1-1 mm, preferably about 0.3 mm, 0.4 mm, 0.5 mm, 0.6 mm, or about 0.7 mm. When the binding particles are paramagnetic silica beads, they may be of diameter 0.5-5 μm, preferably about 0.8 μm, 0.9 μm, 1.0 μm, 1.1 μm or about 1.2 μm.

The apparatus may be engageable with a processing device, preferably an automated device such as an FL instrument or “Maxwell®” machine, which comprises a magnetic “wand” which may be used to move paramagnetic particles, typically in complex with an analyte of interest, from one location to another. Such a magnetic wand may comprise a magnetisable sheath, so that the magnetic properties of the sheath may be reversible by insertion or removal of an elongate magnet into the sheath. An example of such an arrangement is described in detail on page 19 of WO2005/019836. Such a sheath/magnet arrangement may, therefore, act as the grinding element and/or the removal device mentioned above in relation to the first aspect of the invention.

The apparatus may comprise at least one additional chamber (an analyte processing chamber), each of which may optionally comprise further reagents useful in processing and/or analysing the analyte. For example, where the analyte is a nucleic acid, the apparatus may comprise chambers containing wash solutions and elution buffers and additional chambers containing one or more reagents required for a nucleic acid amplification reaction, such as a polymerase chain reaction, to be carried out. Such reagents may include, for example, a polymerase. Alternatively or additionally, the reagents may include one or more nucleic acid primers and/or probes, which may be labelled with one or more detectable labels such as may be required, for example, to enable FRET detection of labels. The skilled person will readily be able to envisage possible reagents which might be present in an additional chamber, according to the requirements of a particular analysis or other procedure to be carried out.

According to a third aspect of the invention, there is provided a kit for use in extracting a nucleic acid analyte from a cell in a sample, preferably according to the first aspect of the invention, comprising free disruption beads (which may comprise external silica or glass), free binding particles comprising external silica or glass and a chaotropic agent such as guanidinium thiocyanate, guanidinium isothiocyanate or guanidinium hydrochloride. The disruption beads may be glass beads, for example of diameter 0.1-1 mm, preferably about 0.3 mm, 0.4 mm, 0.5 mm, 0.6 mm, or about 0.7 mm. The binding particles may be paramagnetic silica beads, for example of diameter 0.1-5 μm, preferably about 0.8 μm, 0.9 μm, 1.0 μm, 1.1 μm or about 1.2 μm.

A related aspect of the invention provides the use of the apparatus according to the second aspect of the invention and/or the kit according to the third aspect of the invention in a method according to the first aspect of the invention.

Throughout the description and claims of this specification, the words “comprise” and “contain” and variations of the words, for example “comprising” and “comprises”, mean “including but not limited to” and do not exclude other moieties, additives, components or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

### BRIEF DESCRIPTION OF THE FIGURES

Embodiments of the invention will now be described, by way of example only, with reference to the following FIGS. 1-8 in which:

FIG. 1 shows a sample chamber (1) containing glass disruption beads (5) and a chaotrophic agent (10) in the form of a dry powder;

FIG. 2 shows a sample (15) being added (downward arrow);

FIG. 3 shows a wand (20) containing an internal soft iron rod (30) with a magnet (25) at the bottom end, the wand having flanges (35) at the top end for manipulation;

FIG. 4 shows the wand (20), after having been withdrawn from the sample chamber an moved to a separate chamber to collect magnetic beads (40), being lowered back into the sample chamber;

FIG. 5 shows the iron rod (30) and magnet having been withdrawn, so that the wand (20) can be used to mix the contents of the chamber (up/down arrow);

FIG. 6 shows the wand (20) being withdrawn (upward arrow) and the iron rod (30) and magnet being re-inserted into the wand (downward arrow);

FIG. 7 shows the wand (20) being moved slowly up and down through the sample (up/down arrow) to allow the magnetic beads to associate with the surface of the wand; and

FIG. 8 shows the wand (20) being removed from the chamber with magnetic beads (40) associated with the wand's surface.

### EXAMPLES

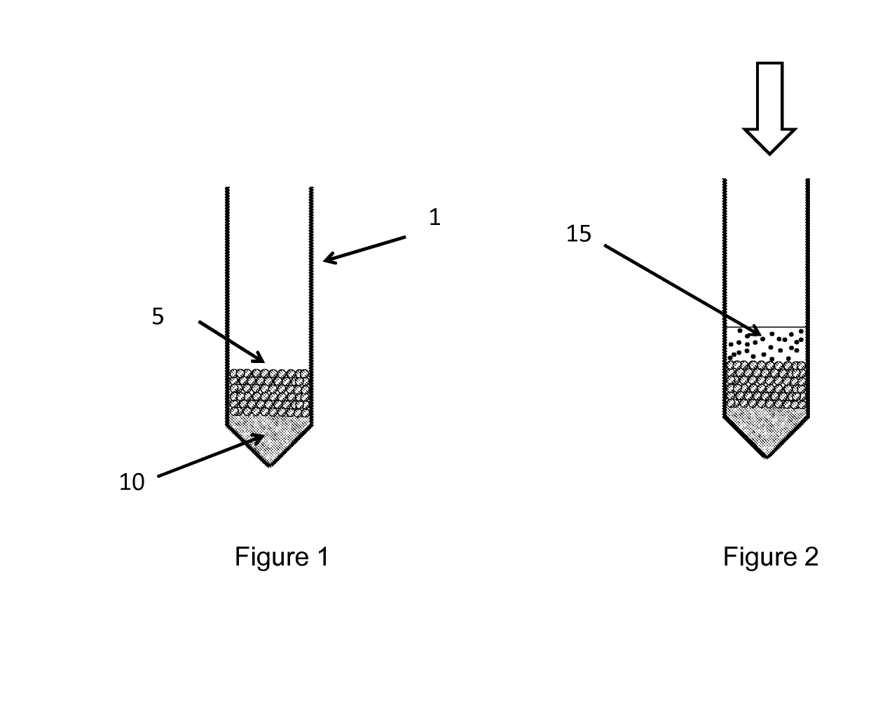

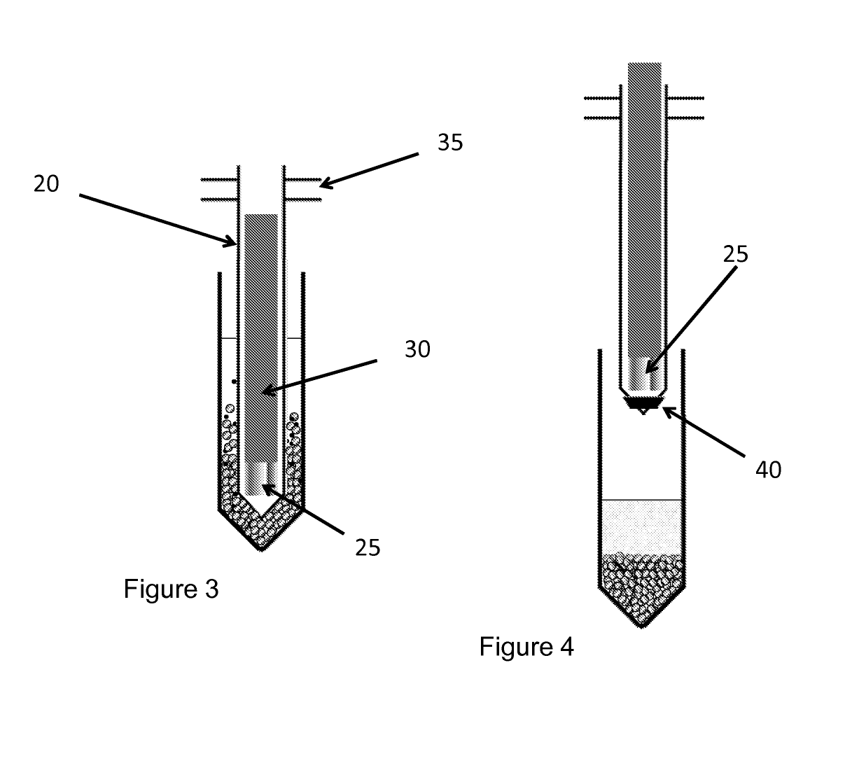

By way of general description with reference to the Figures, FIG. 1 shows a sample chamber (1) provided with glass beads (5) for disruption by grinding and chaotropic agent pre-added as a dry powder (10) which, for aqueous samples, avoids unnecessary dilution. FIG. 2 shows the sample, here spores in aqueous suspension, added to the pre-loaded mixture of glass beads and chaotrope. FIG. 3 shows the wand (20) of the apparatus, which is formed by an outer sheath surrounding a soft iron core (30) which has a magnet (25) at the lower end. The wand (20) has flanges (35) at the upper end to enable manipulation within the sample chamber (1).

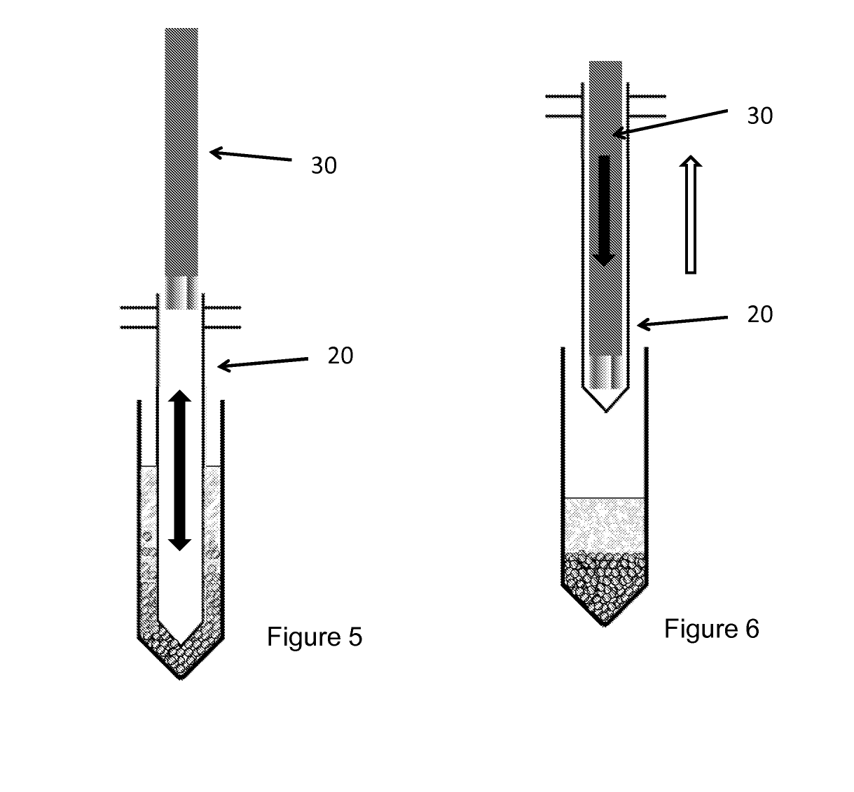

In FIG. 3, the wand (20) is moved up and down through the glass bead/chaotrope/sample mixture. The glass beads are thus ground against one another and against the internal surface of the chamber and physically disrupt the spores and cells in the sample. The internal soft iron core (30) improves the rigidity of the wand (20). After this step, the wand (20) is withdrawn from the sample chamber (1) and is transferred to a separate chamber containing silica-coated magnetic particles (40). The presence of the magnet (25), at the end of the iron core (30), inside the wand (20) causes the magnetic particles (40) to associate with the external surface of the wand (20). The wand (20) is then re-inserted back into the sample chamber (1), taking the magnetic particles (40) with it, as shown in FIG. 4. The magnet (25) and soft iron core (30) is then removed from the wand (20) so that the particles (40) are released from the surface of the wand (20) into the sample mixture. FIG. 5 shows that the wand (20) can then be moved up and down (up/down arrow) so as to mix the sample and magnetic particles together. As a result of the presence of the chaotrope, any nucleic acid present in the sample can associate with the magnetic particles, as they are silica-coated.

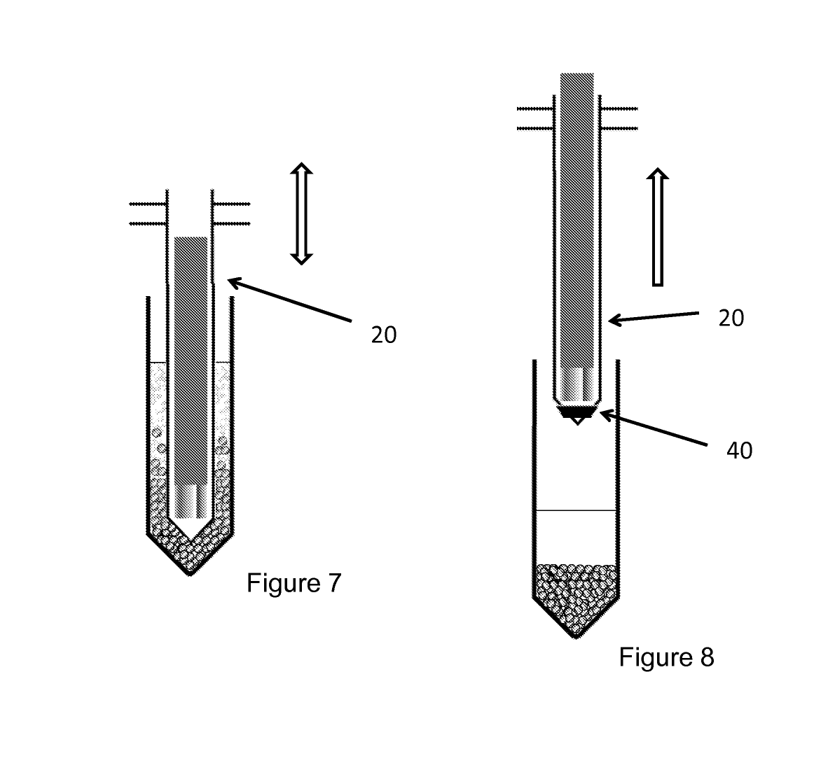

FIG. 6 shows that the wand (20) is removed from the sample chamber (upward arrow) and the soft iron core (30) with the magnet (35) at one end re-inserted into the wand (20) (downward arrow). As shown in FIG. 7, the wand (20) is then slowly moved up and down through the sample, to allow time for the magnetic particles (40), now with any nucleic acid bound, to associate with the external surface of the wand (20) as a result of the presence in the wand of the magnet (25). The wand (20) is withdrawn from the sample chamber (FIG. 8), thereby removing the magnetic particles (40) with bound nucleic acid. The magnetic particles are then ready for transfer to subsequence steps in the process, initially including washes to remove sample matrix and chaotrope.

In specific example of the use of such a system, 500 μl of spore suspension was added to the sample well of an Enigma FL cartridge. This was pre-dosed with guanidinium dry lysis reagent (260 mg guanidinium thiocyanate plus 2.5 mg sodium deoxycholate) and 500 μl of 0.5 mm diameter soda lime glass beads from BioSpec Products Inc. (Cat#11079105). On addition of the sample, the effective concentration of guanidinium thiocyanate was around 4M. The cartridge was loaded into an Enigma FL instrument that was programmed to pick up the wand, move it to the sample and to pound up and down into the sample/lysis/bead mixture 300 times with a stroke length of 10 mm, stopping 6 mm from the bottom of the well. The duration of this process was 70 seconds. Magnetic beads were then transferred from the first wash solution, where they were stored, into the lysis mix in the sample well using the wand/magnet assembly. Normal sample processing of the extracted DNA on the Enigma FL instrument was then carried out with a DNA binding step, three washes of the magnetic beads, followed by elution of the DNA from them. The eluted DNA was then analysed by real-time TaqMan PCR in a Cepheid SmartCycler using 47 μl of eluent, 1 μl each of the two primers and the probe, to give 50 μl in total, to which a freeze-dried Cepheid SmartBead mastermix was added. Thermal cycling parameters were set for 50 cycles of 95° C.×10 sec and 60° C.×10 sec with automated Ct threshold calling.

The results are shown in the Table, showing PCR results for Aspergillus fumigatus under various conditions. With standard chaotropic lysis using guanidinium, no amplification was observed for the spore preparation (which did not contain measurable extracellular DNA) or the low concentration aerosol samples. Following sample processing utilising the cell disruption obtained by grinding with glass beads, sufficient DNA was extracted to allow detection of the spores. Results from PCR of pre-extracted Aspergillus fumigatus DNA are included for reference.

| | | Guanidinium | Guanidinium |
|---|---|---|---|
| | | plus bead | plus bead |
| | Guanidinium | grinding | grinding |
| | extraction only | Run 1 | Run 2 |
| Sample | Ct value | Ct value | Ct value |
| (0.5 ml tested) | (cycle number) | (cycle number) | (cycle number) |
| Aerosol sample with | ND | 39.11 | NEG |
| 80 spores per ml | | | |
| Aerosol sample with | NEG | 36.21 | 36.16 |
| 8,000 spores per ml | | | |
| Aerosol sample with | 36.71 | 31.08 | 31.76 |
| 744,000 spores per ml | | | |
| Aspergillus spore | NEG | 30.60 | 30.02 |
| preparation with | | | |
| 40,000 spores | | | |
| per ml | | | |
| 1 ng Aspergillus DNA | ND | 28.72 | ND |
| 10 ng Aspergillus | ND | 25.53 | ND |
| DNA | | | |
| 20 ng Aspergillus | ND | 24.87 | ND |
| DNA | | | |

ND = Not Determined
NEG = no amplification observed

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

1. A method of extracting a nucleic acid analyte from a cell or virus in a sample chamber, comprising
adding disruption beads comprising external silica or glass to the sample chamber;
agitating the disruption beads within the sample chamber to disrupt the cell;
adding binding particles comprising external silica or glass to the sample chamber in the presence of a chaotropic agent;
contacting the contents of the sample chamber with a removal device with which the binding particles reversibly associate; and
separating the removal device and associated binding particles from the sample chamber, thereby removing nucleic acid analyte from the sample.

2. The method of claim 1 wherein the chaotropic agent is guanidinium thiocyanate, guanidinium isothiocyanate or guanidinium hydrochloride.

3. The method of claim 1 wherein the agitation in step (b) comprises grinding the disruption beads in the chamber using a grinding element.

4. The method of claim 1 wherein the cell is a bacterial, plant, animal or a fungal cell or a virus particle.

5. The method of claim 1 wherein the cell is an Aspergillus fumigatus cell.

6. The method of claim 1 wherein the disruption beads are glass beads.

7. The method of claim 1 wherein the binding particles are magnetic silica beads and the removal device is a magnet.

8. The method of claim 1 wherein the disruption beads are glass beads of diameter 0.1-1 mm and the binding particles are magnetic silica beads of diameter 0.1-5 μm.

9. An apparatus for use in a method of extracting an analyte from a cell in a sample, comprising:
a first chamber containing disruption beads comprising external silica or glass, and
a chaotropic agent and binding particles comprising external silica or glass;
wherein the chaotropic agent and/or binding particles are contained in the first chamber and/or in a second chamber.

10. The apparatus of claim 9 further comprising at least one analyte processing chamber.

11. The apparatus of claim 9 wherein the disruption beads are glass beads.

12. The apparatus of claim 9 wherein the binding particles are paramagnetic silica beads.

13. The apparatus of claim 9 wherein the disruption beads are glass beads of diameter 0.1-1 mm and the binding particles are paramagnetic silica beads of diameter 0.1-5 μm.

14. A kit for use in extracting an analyte from a cell in a sample, comprising disruption beads comprising external silica or glass, binding particles comprising external silica or glass and a chaotropic agent.

15. The kit of claim 14 wherein the disruption beads are glass beads.

16. The kit of claim 14 wherein the binding particles are paramagnetic silica beads.

17. The kit of claim 14 wherein the disruption beads are glass beads of diameter 0.1-1 mm and the binding particles are para magnetic silica beads of diameter 0.1-5 μm.

18. (canceled)
